(19) United States
(12) Patent Application Publication
(10) Pub. No.: US 2024/0125727 A1
(43) Pub. Date: Apr. 18, 2024

(54) ELECTROCHEMICAL MEASUREMENT WITH ADDITIONAL REFERENCE MEASUREMENT

(71) Applicant: Radiometer Medical ApS, Brønshøj (DK)

(72) Inventors: Thomas KJAER, Brønshøj (DK); Poul Ravn SØRENSEN, Brønshøj (DK)

(21) Appl. No.: 18/263,387

(22) PCT Filed: Jan. 28, 2022
(86) PCT No.: PCT/EP2022/052010

§ 371 Date: Jul. 28, 2023

(30) Foreign Application Priority Data
Jan. 29, 2021 (EP) 21154377.2

(51) Int. Cl. G01N 27/27; G01N 27/333; G01N 33/84

(52) U.S. Cl. CPC G01N 27/27; G01N 27/333; G01N 33/84

(57) ABSTRACT

There is presented a method of measuring one or more potential differences being indicative of one or more concentrations of one or more analyte ions in a sample, such as being a liquid whole blood sample, said method comprising, measuring with a reference ion measurement setup a parameter indicative of a concentration of a reference ion in the sample wherein the reference ion measurement setup is different from an electroanalytical measurement setup, measuring with an analyte ion measurement setup one or more potential differences directly or indirectly between each of one or more, optionally solid-state, working electrodes with each of said, optionally solid-state, working electrodes comprising an ion-selective electrode which is selective for an analyte ion, and an, optionally solid state, reference electrode which is selective for the reference ion wherein the analyte ion measurement setup is an electroanalytical setup. There is additionally presented an apparatus and use of said apparatus.

### FIELD OF THE INVENTION

The present invention relates to a method of measuring a parameter indicative of a concentration of an analyte ion in a liquid, and more particularly relates to a method for measuring one or more potential differences being indicative of one or more concentrations of one or more analyte ions in a sample, such as the sample being a liquid whole blood sample, and furthermore relates to a corresponding apparatus and use of such apparatus.

### BACKGROUND OF THE INVENTION

It is generally advantageous for numerous reasons to be able to determine one or more potential differences being indicative of one or more concentrations of one or more analyte ions in a sample, such as the sample being a liquid whole blood sample, yet doing so might involve costly, complicated and/or inaccurate equipment, which may also require a large sample volume, and which may additionally have limited service life and/or be prone to breakdown.

Therefore, there is a need for an improved method of and apparatus for measuring one or more potential differences being indicative of one or more concentrations of one or more analyte ions in a sample, such as the sample being a liquid whole blood sample, and in particular an improved method and apparatus which is more cost efficient, simpler, more accurate, enables working with smaller sample volumes, and which may additionally be more durable and/or being less prone to breakdown.

### SUMMARY OF THE INVENTION

An object of the present invention is to provide an improved method and apparatus overcoming at least some of the disadvantages of known methods of and apparatuses for measuring one or more potential differences being indicative of one or more concentrations of one or more analyte ions in a sample, such as the sample being a liquid whole blood sample. Additionally, or alternatively, it is an object of the present invention to provide an alternative to known methods and apparatuses.

According to a first aspect, the invention provides a method of measuring one or more potential differences being indicative of one or more concentrations of one or more analyte ions in a sample, such as the sample being a liquid whole blood sample, and optionally furthermore determining the one or more concentrations of the one or more analyte ions, said method comprising,
Optionally providing an apparatus according to the second aspect,
Measuring with a reference ion measurement setup a parameter indicative of a concentration of a reference ion in the sample, wherein the reference ion measurement setup (104) is different from an electroanalytical measurement setup, and optionally furthermore determining the reference ion concentration, and
Measuring with an analyte ion measurement setup the one or more potential differences being indicative of the one or more concentrations of the one or more analyte ions in the sample directly or indirectly between:
   i. each of one or more working electrodes, such as one or more solid-state working electrodes, each of said working electrodes comprising an ion-selective electrode, which is selective for an analyte ion, and
   ii. a reference electrode, such as a solid-state reference electrode, which is selective for the reference ion,
wherein the analyte ion measurement setup is an electroanalytical setup, such as a potentiometric setup,
and optionally determining the one or more concentrations of the one or more analyte ions.

A possible advantage of the present invention is that the reference ion measurement setup enables dispensing with the need of a reference electrode of known or predictably varying potential, such as dispensing with the need of a reference with a stable potential or dispensing with the need of knowing or being able to predict conditions for a reference with a potential varying with conditions, which may in turn yield one or more of a more cost efficient, simpler and/or more accurate method, which may additionally be more durable and/or being less prone to breakdown, and/or may enable working with smaller sample volumes.

A gist of the invention may be seen as realizing that providing a constant, known or predictably varying potential may advantageously be replaced with a measurement of a concentration of reference ion, which would diminish the requirements of the reference electrode (e.g., by not requiring that the electrode maintained a constant potential) and/or the requirements of the measurement solution, such as liquid whole blood (e.g., by not requiring a parameter, such as a concentration of reference ion, of the measurement solution to be known or predictable).

For example, instead of relying on a reference electrode of nominally known potential by means of an aqueous electrode, such as standard hydrogen electrode (SHE), it may be possible to have as reference a solid-state ion-selective electrode, such as a pH sensitive solid-state electrode (and additionally have a reference measurement setup for measuring pH). Aqueous reference electrodes may be circumstantial, expensive, space consuming (e.g., due to the liquid-liquid junction and separate electrolyte solution chamber) and may have limited service life due to loss of ions from the electrolyte chamber to the sample and/or rinse solution. Furthermore, the ionic loss itself may cause a problem of contamination of sample and/or rinsing solutions, which may introduce measurement errors and loss of accuracy. Leaking ions of an aqueous reference electrode may necessitate a large distance between working electrode and reference electrode, which may in turn increase size and necessitate larger sample volumes (where large volumes may be particularly disadvantageous for whole blood samples in certain cases, such as for neonates and/or intensive care patients).

As another example, instead of relying on a reference electrode varying with conditions and furthermore relying on knowing or predicting the measurement conditions, it may be possible to have as reference an ion-selective electrode, such as a pH sensitive solid-state electrode, and measure the reference ion concentration, such as via a setup for measuring pH. An advantage of measuring the reference ion concentration may be that it enables increased accuracy of determination of a concentration of an analyte ion, where the increase in accuracy of determination of the concentration of the analyte ion comes from the increase in accuracy of the concentration of the reference ion due to a measurement being more accurate than an assumption or a prediction. Additionally, it may be seen as an advantage of measuring the reference ion concentration that it expands the usability of the method to applications where conditions, such as concentrations of reference ions, are not known or predictable. For example, when relying on known, constant concentration of a specific reference ion or a situation where a concentration of a specific reference ion behaves in a predictable manner, the choice of reference ion is limited to reference ion (candidates) where a concentration is known and constant or behaves in a predictable manner. This may be disadvantageous for several reasons, including that no (reference) ion concentration is truly constant across individuals (such as for different humans) and/or that there might not be overlap between the thus applicable reference ions and the optimal ion selective reference electrode (in other words, it might be necessary to employ a less-than-optimal reference electrode, because the concentration of the reference ion for which the optimal reference electrode is specific is not known and constant or predictable). In contrast, in embodiments according to the present invention, the reference ion may be freely chosen, e.g., in accordance with the optimal reference (ion specific) electrode. This might in turn benefit the accuracy of the reference ion measurement setup, which might in turn benefit the overall accuracy with respect to determination of the analyte ion concentration.

By ‘measuring one or more potential differences’ is to be understood as is common in the art (where potential differences are electrical potential differences), such as measuring one or more voltages, such as DC voltages. Measuring such potential difference may be done, e.g., by potentiometry. It is understood that in case of multiple potential differences, each potential difference is to be measured between a reference electrode and a (respective) working electrode.

By ‘being indicative of one or more concentrations of one or more analyte ions in a sample’ may be understood, that the method is arranged so that the measured potential differences are each indicative of a concentration of an ion or a group of ions in the sample.

By ‘indicative of a concentration’ may in general be understood that a concentration may be determined therefrom. For example, a potential difference—which is not in itself a concentration—is measured and it enables determining the concentration. By determining concentration may be understood both qualitatively detecting a presence (yes/no) of an analyte, such as the concentration exceeding a detection limit, and quantitatively determining a concentration, such as on an ordinal, interval or ratio type scale.

By ‘reference ion’ is understood any ion, possibly naturally present in whole blood, applicable as a reference ion in the sense that a (reference) electrode selective for said (reference) ion can be used as a reference electrode, such as a reference electrode in a potentiometric setup. The reference ion could for example be any of the hydrogen ion (H⁺), the sodium ion (Na⁺), the potassium ion (K⁺), the calcium ion (Ca²⁺), the chloride ion (Cl⁻), the magnesium ion (Mg²⁺) or the bicarbonate or hydrogen carbonate ion (HCO₃⁻).

By ‘reference ion measurement setup’ is understood any setup capable of determining a parameter indicative of a concentration of a reference ion (wherein it is generally understood that the reference ion may be understood to be a single, specific reference ion, or a group of reference ions), such as a setup arranged for optically probing the sample, such as the whole blood sample, and determining an optical parameter thereof being indicative of the reference ion concentration.

The reference ion measurement setup may be selective for a single ion or a group of ions.

The reference ion measurement setup be somewhat (cross-)selective to one or more interfering ions. However, the reference ion measurement setup may be understood to be more selective to an ion or a group of ions compared to another ion or group of ions (such as a selectivity coefficient of the interfering ions being less than 1.0, such as less than 0.9, such as less than 0.5, such as less than 0.1, such as less than 0.07, such as less than 0.05, such as less than 0.03, such as less than 0.02, such as less than 0.01).

It may in general be advantageous with negligible or zero cross-selectivity, e.g., since it enables dispensing with a need for taking a cross-selectivity into account altogether. If cross-selectivity is non-zero, such as non-negligible, it may still be advantageous if cross-selectivity is below a certain threshold, such as a selectivity coefficient of the interfering ions being less than 1.0, such as less than 0.9, such as less than 0.5, such as less than 0.1, such as less than 0.07, such as less than 0.05, such as less than 0.03, such as less than 0.02, such as less than 0.01, e.g., because if cross-selectivity is sufficiently low (such as a selectivity coefficient of the interfering ions being less than 1.0) it may still be possible to make a correction and take a cross-selectivity into account and still be able to utilize the measurement.

According to an embodiment the reference ion is a single, specific ion or a group of ions and there is practically no cross-selectivity in either of the reference ion measurement setup or the reference electrode.

According to another embodiment, the reference ion is a single, specific ion or a group of ions and there is cross-selectivity in the reference ion measurement setup and/or the reference electrode and each cross-selectivity is accounted for, e.g., by measuring and/or estimating a concentration of each of the one or more interfering species and taking these one or more concentrations into account. ‘Electroanalytical measurement setup’ is understood as is common in the art, such as a setup an electroanalytical method, such as wherein an ‘electroanalytical method’ is a method for chemical analysis, such as a method enabling determining a parameter indicative of a concentration of an reference ion and/or an analyte ion in a liquid sample, by an electrolytic method, where ‘electrolytic method’ is understood as is common in the art, such as a method for producing chemical changes by application of an electrical potential and/or passage of an electric current through an electrolyte. More particularly, it is understood that an electroanalytical method determines the parameter indicative of concentration of the analyte ion and/or the reference ion by measuring an electrical potential (volts) and/or current (amperes) in an electrochemical cell containing the analyte ion and/or reference ion. An electroanalytical method uses electrically conductive probes, called electrodes, to make electrical contact with the analyte solution. The electrodes are used in conjunction with electric or electronic devices to which they are attached to measure the parameter of the solution. The measured parameter is related to the identity of the analyte ion or reference ion and/or to the quantity of the analyte ion or reference ion in the solution. Electroanalytical methods include potentiometry, amperometry, conductometry, electrogravimetry, voltammetry (and polarography), and coulometry.

By ‘the reference ion measurement setup is different from an electroanalytical measurement setup’ is understood that the reference ion measurement setup is present and applied in the method, yet belongs to a group of measurement setups being different with respect to the group of electroanalytical measurements setups. An advantage of this may be that while electroanalytical measurement setups generally require a reference electrode, this requirement can be dispensed with for measurements setups different with respect to electroanalytical measurement setups. Additionally, once the parameter indicative of a concentration of a reference ion in the sample has been determined, it then becomes possible to benefit from the advantages of an electroanalytical method (such as potentiometry) for the purpose of determining parameters, i.e., the potential differences, being indicative of the one or more concentrations of the one or more analyte ions in the sample without requiring a reference electrode with a predetermined and known potential.

In some embodiments the reference ion measurement setup is chosen from a group of measurements setups relying on measuring an optical parameter (such as emitted light and/or an optical property of the sample probed with light, such as fluorescence, chemiluminescence, refractive index or absorption), a mass parameter (e.g., with a Quartz-Crystal Microbalance (QCM)), a magnetic field parameter (e.g., with a Hall sensor in conjunction with labelled magnetic nanoparticles), a stress parameter (e.g., with a microcantilever) or a dissipation parameter (with e.g., a Quartz-Crystal Microbalance with dissipation mode (QCM-D)).

For example, the reference ion setup could be a setup for determining the reference ion concentration of the sample, such as the whole blood sample, via optical probing of the sample, such as an optical pH sensor. ‘Optical probing’ is understood as is common in the art, such as relating to irradiating light onto at least a portion of the sample and receiving at least a portion of (luminescent) light therefrom, where the received light enables deriving information (such as a concentration) about an analyte (such as a reference ion) therein. In another example, the reference ion measurement setup could be a setup based on measurement of mechanical properties (such as dissipation) and/or mass, such as with a Quartz-Crystal Microbalance sensor with dissipation measuring capabilities (QCM-D) measuring mass and/or dissipation, or measuring mass (in dynamic mode) and/or stress (in static mode) with a microcantilever, or a setup based on measurement of a magnetic parameter, such as measuring magnetic field of labelled magnetic particles with a Hall-sensor. It is encompassed that a parameter indicative of a concentration of the reference ion may be measured directly or indirectly since it is encompassed that an effect, such as a conformational change in macromolecules induced by the reference ion or such as an adsorption rate or desorption rate of relatively heavier molecules from a surface being affected by the reference ion, is measured and wherefrom an indirect measure of concentration of the reference ion can be derived.

In general, when referring to ‘optic’, ‘optical’, etc., throughout this application, it may generally be understood to be done with reference to electromagnetic radiation, such as light with wavelength(s) within the range from 380 nm to 750 nm.

In some embodiments, the reference ion measurement setup is a setup relying on a measurement principle different from electroanalytical techniques, such as said different measurement principle relying on another effect (e.g., a change in optical properties, mechanical properties or mass) than an effect (such as potential or current) measurable with an electroanalytical technique (such as potentiometry, coulometry, voltammetry). A possible advantage of this might be that it reduces or eliminates a risk of damaging the sample, such as a risk of extracting ions from red blood cells, as might be a risk of, e.g., chronopotentiometry. Another possible advantage is that it dispenses with the need of a reference electrode of a known potential.

By ‘Measuring with a reference ion measurement setup a parameter indicative of a concentration of a reference ion in the sample’ is understood that a parameter is determined by means of the reference ion measurement setup, which parameter enables determining the concentration of the reference ion in the sample, for example wherein said parameter is an intensity of (luminescent) light within a certain wavelength.

By ‘Measuring one or more potential differences directly or indirectly’ may be understood, that each potential difference may be determined directly between two points, such as directly between a working electrode and a reference electrode, such as via a (optionally high-impedance) voltmeter directly between the working electrode and the reference electrode, or indirectly, such as each of the potential differences between a working electrode and the reference electrode are measured with respect to a third electrode, and the potential difference between the working electrode and the reference electrode is subsequently determined and hence (only) indirectly measured. ‘Working electrode’ is understood as is common in the art, such as an electrode wherein the analyte ion can react and wherein this reaction can be measured.

‘Ion-selective electrode’ (ISE) is understood as is common in the art. The ISE may be selective for a single ion or a group of ions. More particularly, an ISE is an electrochemical sensor or electrode that allow potentiometric determination of the activity of a certain ion in the presence of other ions. An ISE may comprise an ion-selective membrane allowing passage of only (taking into account possibly some cross-selectivity as described in the following) the selected ion to a conducting, internal electrode. ISEs might encompass electrodes which are somewhat (cross-)selective to one or more interfering ions. However, an ISE may be understood to be more selective to an ion or a group of ions compared to another ion or group of ions (such as a selectivity coefficient of the interfering ions being less than 1.0, such as less than 0.9, such as less than 0.5, such as less than 0.1, such as less than 0.07, such as less than 0.05, such as less than 0.03, such as less than 0.02, such as less than 0.01).

The ‘membrane’ is fully or partially solid but may also contain a plasticiser (such as wherein the remainder is partially or fully a liquid), such as comprises at least 20 volume/volume percent (v/v %) solid matter, such as comprises at least 40 v/v % solid matter, such as comprises at least 60 v/v % solid matter, such as comprises at least 80 v/v % solid matter such as comprises at least 90 v/v % solid matter, such as comprises at least 95 v/v % solid matter, such as comprises at least 99 v/v % solid matter, such as is fully solid.

‘Reference electrode’ is understood as is common in the art, such as an electrode which can be used as a point of reference for measurement of the potential difference with respect to each of the one or more working electrodes.

The reference electrode may be somewhat (cross-)selective to one or more interfering ions. However, the reference electrode may be understood to be more selective to an ion or a group of ions compared to another ion or group of ions (such as a selectivity coefficient of the interfering ions being less than 1.0, such as less than 0.9, such as less than 0.5, such as less than 0.1, such as less than 0.07, such as less than 0.05, such as less than 0.03, such as less than 0.02, such as less than 0.01).

By ‘analyte ion’ may be understood an ion whose concentration is of interested and/or to be determined.

According to some embodiments, there is presented a method, wherein each of the one or more working electrodes are solid-state (ion-selective, working) electrodes and/or wherein the reference electrode is a solid-state (ion-selective, reference) electrode. A ‘solid-state’ electrode is understood as is common in the art. More particularly, by solid-state ion-selective electrode is understood an ion-selective electrode comprising an ion-selective membrane and a conducting, internal electrode, wherein little or no liquid is present between the membrane and the electrically conducting, internal electrode, such as between a side of the membrane facing the conducting, internal electrode and a side of the conducting, internal electrode facing the membrane. By ‘little’ liquid may be understood a volume of liquid being less than 10 times a volume of the membrane, such as less than 5 times a volume of the membrane, such as less than 2 times a volume of the membrane, such as less than a volume of the membrane, such as less than 0.5 times a volume of the membrane, such as less than 0.1 times a volume of the membrane, such as less than 0.01 times a volume of the membrane. In addition or alternatively, it may be understood, that the conducting, internal electrode is in contact with or close to the membrane, such as a percentage of a distance from the conducting, internal electrode to the opposite side of the membrane being given by a distance from the conducting, internal electrode to the membrane being less than 90%, such as less than 75%, such as less than 50%, such as less than 25%, such as less than 10%, such as less than 5%, such as less than 2%, such as less than 1%, such as less than 0.1%. In addition, or alternatively, it may be understood, that the conducting, internal electrode is arranged so as to enable being close or coming closely to the sample, such as a distance between the conducting, internal electrode and the sample being less than 1 mm, such as less than 0.75 mm, such as less than 0.5 mm, such as less than 0.25 mm, such as less than 0.1 mm, such as less than 0.03 mm, such as less than 0.01 mm, such as less than 0.003 mm.

By ‘conducting, internal electrode’ may be understood the sub-part of the solid-state whereupon the electrochemical reactions occur, and which is electrically conductive and coupled to analysis equipment, such as a (high-impedance) voltmeter. It is thus understood that the (electrically) conducting, internal electrode forms part of the solid-state electrode. The conducting electrode may comprise one or more different materials, where one or more of the different materials might comprise non-solid matter, such as liquid, and/or be susceptible to liquid uptake, such as wherein the conducting internal electrode forms a matrix comprising solid material. The conducting, internal electrode is fully or partially solid (such as wherein the remainder is partially or fully liquid), such as comprises at least 50 v/v % solid matter, such as comprises at least 60 v/v % solid matter, such as comprises at least 70 v/v % solid matter, such as comprises at least 80 v/v % solid matter, such as comprises at least 90 v/v % solid matter, such as comprises at least 95 v/v % solid matter, such as comprises at least 99 v/v % solid matter, such as is fully solid.

According to an embodiment, there is presented a method wherein each of the one or more working electrodes are solid-state electrodes and wherein the reference electrode is a solid-state reference electrode. A possible advantage of this may be that disadvantages associated with non-solid (aqueous or liquid-liquid) electrodes are overcome, such as disadvantages associated with large size, large costs, electrolyte being contaminated or consumed, fragility and/or erroneous measurements, e.g., due to electrolyte leaking (out of an aqueous, liquid-liquid electrode) and affecting measurements (at the working electrode).

According to an embodiment, there is presented a method comprising determining one or more concentrations of the one or more analyte ions in the sample based on:
i. The concentration of the reference ion, and
ii. The one or more potential differences.

An advantage of this may be that the one or more concentrations of the one or more analyte ions are determined, and/or that said determination(s) can be done, with high(er) accuracy due to the concentration of the reference ion (as determined with the reference ion measurement setup) being taken into account, such as not only relying on an assumed—and possible sought (e.g., by addition of a certain amount of the reference ion)—concentration of the reference ion. Determining a concentration of an analyte ion may be done by calculating the concentration, such as by utilizing the Nernst equation.

For the purpose of determining the one or more concentrations of the one or more analyte ions in the sample, the following framework may be relied upon.

The Nernst equation gives a relation between potential and concentration of an analyte ion:

E=E0+N_fac*log₁₀ (X), where N_fac is the slope (Nernst factor) of the half-cell, e.g., where X=cH⁺, cK⁺, cNa⁺, cCa⁺⁺ or cCl⁻ (where the ‘c’ indicates ‘concentration of’). In the following “+” and “−” may be omitted, e.g., cK means cK⁺, i.e., the concentration of the potassium ion; where an element, such as K is without prefix c, it is just the parameter K (potassium). By a difference between samples, E₀ is eliminated.

For the example of pH: ΔE(pH)=N_fac (pH)*log₁₀ (cH1/cH2), where cH1=cH (i.e., concentration of the hydrogen ion, H⁺) for sample 1 and cH2=cH for sample 2.

In the same way for electrolytes, e.g., for cK: ΔE(cK)=N_fac (cK)*log₁₀ (cK1/cK2)

An equation can be developed for N_fac(pH)=Nernst factor for parameter pH, and similarly for N_fac(X): Nernst factor for the X parameter, where X denotes the electrolytes. These equations are to be used by calculation of a correction potential (see below), resp., by calculation of the concentration of the analyte in the sample.

Derivation of equation for N_fac(X): Between sample 1 to sample 2 with pH as reference:

$$Δ E ( 12 X ) pH = Δ E ( X ) - Δ E ( pH ) = N\_fac ( X ) * {log}_{10} ( \frac{X 1}{X 2} ) - N\_fac ( pH ) * {log}_{10} ( \frac{H 1}{H 2} )$$

Which can be reformulated into:

$$N\_fac ( X ) = ( Δ E ( 12 X ) pH + N\_fac ( pH ) * {log}_{10} ( \frac{H 1}{H 2} ) ) / {log}_{10} ( \frac{X 1}{X 2} )$$

By calculation of N_fac(X) the value of N_fac(pH) can be estimated to, e.g., 57 mV (e.g., based on prior knowledge about the slope of the half-cell of the electrode). A set of optionally optimal calibrating solutions (“best”), such as solutions with the same or nearly the same pH value, may be used for obtaining N_fac(pH) with the following formula:

$$N\_fac ( pH ) = ( N\_fac ( X ) best * {log}_{10} ( \frac{X 1}{X 2} ) - Δ E ( 12 X ) pH ) / {log}_{10} ( \frac{H 1}{H 2} )$$

The “best” may refer to N_fac(X) for the set of optionally optimal solutions, such as the two solutions with the same nearly the same pH value, where—for n_Fac(pH) the mean value of the two values is calculated and used in further calculations.

Since pH for an (unknown) sample is unknown and possibly different with respect to a calibration solution, this pH difference has to be calculated into a voltage (a millivolt (mV) value), which is subtracted by calculation of the different electrolyte concentrations. The applicable formula, such as for calculation of the correction potential Pot.corr, is:

Pot.corr=ΔpH*N_fac (pH)

A new potential can be calculated for an example where ‘cal1’ is a calibration liquid and ‘sample’ is an unknown sample, whose concentration of analyte ion is to be determined:

E(new)=ΔE((cal1−sample)X)pH−Pot.corr.

The electrolyte concentration (mM) can be calculated as:

$$cXsample = \frac{cXcal 1}{\frac{E ( new )}{{10}^{N\_fac ( X )}}}$$

According to an embodiment, there is presented a method, wherein the one or more potential differences depend on the reference ion concentration in the sample. It may thus be understood that the one or more potential differences can depend on the ion concentration in the sample, such as via dispensing with a need for of a reference electrode of known or predictably varying potential. An advantage may be that this need can be dispensed with.

According to an embodiment, there is presented a method wherein determining the one or more concentrations of the one or more analyte ions in the sample is based on an expression reflecting and/or incorporating the dependency of the one or more potential differences on the reference ion concentration in the sample. For example, the reference ion concentration may effect the one or more potential differences as described in the equations above, and this same dependency of the one or more potential differences can be incorporated in the expression for the electrolyte concentration (cf, e.g., the last equation above, expressing ‘cXsample’). In other words, the reference ion concentration is allowed to influence the one or more potential differences in a certain manner (such as with a logarithmic term), but in the expression for determining the one or more concentrations of the one or more analyte ions, this dependency is at least partially, such as partially or fully, taken into account in the same manner, such as reducing, minimizing or eliminating the effect of the reference ion concentration on the (determined) one or more concentrations of the analyte ions in the sample. By ‘reflecting and/or incorporating’ may be understood that the same or similar term (which may be a negative or inverse term for negating an effect) is included in said expression as in (an expression expressing) an effect of the reference ion concentration on the one or more potential differences.

According to an embodiment, there is presented a method, wherein the sample is a liquid whole blood sample. A possible advantage of the sample being a whole blood sample may be that it dispenses with a need to separate fractions of the (original) whole blood sample, such as providing plasma or serum. The term “whole blood” is understood as is common in the art, such as blood where no naturally occurring part has been removed, such as non-processed blood, from (and optionally drawn from) a human or an animal. More particularly, whole blood may refer to blood composed of blood plasma, and cellular components. The plasma represents about 50%-60% of the volume, and cellular components represent about 40%-50% of the volume. The cellular components are erythrocytes (red blood cells), leucocytes (white blood cells), and thrombocytes (platelets). Preferably, the term “whole blood” refers to whole blood of a human subject but may also refer to whole blood of an animal. The terms “blood plasma” or “plasma” refer to the liquid part of the blood and lymphatic liquid, which makes up about half of the volume of blood (e.g., about 50%-60% by volume). Plasma is devoid of cells. It contains all coagulation factors, in particular fibrinogen and comprises about 90%-95% water, by volume. Plasma components include electrolytes, lipid metabolism substances, markers, e.g., for infections or tumors, enzymes, substrates, proteins and further molecular components.

According to an embodiment, there is presented a method comprising aspirating the sample, such as aspirating a sample at a sample inlet of an apparatus, such as an apparatus according to the second aspect, thereby creating an aspirated portion of the sample. A possible advantage of this may be that a controlled portion of sample can be aspirated, such as aspirated and analysed. In a further embodiment, each of measuring with the reference ion measurement setup and measuring with the analyte ion measurement setup is done on the aspirated portion of the sample. A possible advantage of this may be that the (same) aspirated portion of the sample is subjected to both types of analysis, and hence a risk of difference between (sub-)sample portions can be reduced or eliminated.

According to an embodiment, there is presented a method, wherein the reference ion measurement setup is based on a measurement principle based on an optically measurable parameter, such as a reference ion sensitive indicator (changing one or more optical properties, such a absorption (e.g., in a wavelength region of ]400; 1200[ nm, such as in a wavelength region of ]500; 1100[ nm, such as in a wavelength region of ]600; 1000[ nm, such as in a wavelength region of ]700; 900[ nm), depending on a concentration of the reference ion), such as a pH sensitive luminescent indicator, and wherein a measurement principle of the analyte ion measurement setup is a potentiometric measurement principle.

An advantage of this may be that a disadvantage associated with a potentiometric measurement principle necessitating certain electrodes and/or conditions, may be fully or partially overcome, e.g., by using an optically based measurement principle, which in effect may for example dispense with a need of establishing a known potential via a aqueous, liquid-liquid reference electrode.

By ‘measurement principle’ may be understood a principle of measuring a concentration relying on measurement of a certain parameter, such as (electrical) voltage or (optical) absorbance or luminous intensity.

According to an embodiment, there is presented a method, wherein measuring the one or more potential differences involves measuring one or more potential differences exclusively, such as directly, between solid-state electrodes, such as directly between the solid-state working electrode and the solid-state reference electrode. An advantage of this may be that only solid-state electrodes are involved, such as excluding aqueous, liquid-liquid electrodes (and associated disadvantages). By ‘exclusively’ between solid-state electrodes is understood that only potential differences between solid-state electrodes are measured, but the potential difference between the solid-state working electrode and the solid-state reference electrode can be measured as the sum or difference between potential differences between, respectively, the solid-state working electrode and a third solid-state electrode, and the solid-state reference electrode and the third solid-state electrode. By ‘directly’ is understood that the potential difference is measured directly between the solid-state working electrode and the solid-state reference electrode, such as by inserting a (high-impedance) voltmeter between the solid-state working electrode and the solid-state reference electrode.

According to an embodiment, there is presented a method, wherein measuring with the reference ion measurement setup a concentration of the reference ion in the sample, comprises an optical measurement. By an ‘optical measurement’ is understood any measurement, which measures an optical parameter, such as any of absorption rate, reflectance, fluorescence, refractive index, luminous flux, absorption and wavelength. An advantage of an optical measurement might be that it can be implemented without damaging red blood cells. Another possible advantage might be that it is independent from electroanalytical techniques and hence offers an alternative to electroanalytical techniques. Another possible advantage might be that it dispenses with a need of a known electrical potential, such as a known electrical potential as provided by an aqueous, liquid-liquid reference electrode.

According to an embodiment, there is presented a method, wherein the reference ion is the hydrogen ion, such as H⁺, the sodium ion, such as Na⁺, or the potassium ion, such as K⁺. A possible advantage of utilizing the hydrogen ion as reference ion is that it can be assumed present in numerous samples, such as in particular in whole blood samples. A possible advantage of using the sodium ion as reference ion is that it may be present in relatively high concentration (e.g., being a principal ionic constituent in human blood plasma, such as the ionic constituent in human blood plasma of the highest concentration), that it may be measured optically, such as using an (optionally readily available) sodium ion sensitive fluorophore, that the sodium ion concentration correlates with ionic strength (and employing the sodium ion as reference ion will thus include and rule out effects of ionic strength) and/or that it (such as its concentration) may be relatively stable (e.g., the absolute and/or relative fluctuations may be relatively small), e.g., in human blood plasma. A possible advantage of using the potassium ion as reference ion is that it may be present in relatively high concentration (e.g., being a principal ionic constituent in human blood plasma, such as the ionic constituent in human blood plasma of the second-highest concentration).

According to an embodiment, there is presented a method, wherein the optical measurement comprises measuring an optical parameter Pₒ (such as absorption rate, reflectance, fluorescence, refractive index or colour) which is dependent on pH and wherein a change dPₒ in optical parameter with a change dpH in pH, dPₒ/dpH has a local and/or global maximum within a pH interval of [7; 8], such as within a pH interval [7.2, 7.6], such as at or about 7.4. A possible advantage of this is that it a maximum sensitivity might hence be in a region where pH of whole (human) blood is expected. Hence, an accuracy of determination of pH (or hydrogen ion concentration) and hence of determination of the concentrations of the one or more analytes is increased.

According to an embodiment, there is presented a method, wherein the reference ion is an ion naturally occurring within (human) whole blood, such as any of:
a hydrogen ion, such as H⁺,
a sodium ion, such as Na⁺,
a potassium ion, such as K⁺,
a calcium ion, such as Ca²⁺,
a chloride ion, such as Cl⁻,
a magnesium ion, such as Mg²⁺, or
a hydrogen carbonate ion, such as HCO₃.

In this case, the reference ion may be measured directly on whole blood without the need for addition of ions to serve as reference ions.

According to a second aspect, there is presented an apparatus for measuring one or more potential differences being indicative of one or more concentrations of one or more analyte ions in a sample, such as the sample being a liquid whole blood sample, comprising:
a reference ion measurement setup, being arranged for measuring a parameter indicative of a concentration of a reference ion, wherein the reference ion measurement setup (104) is different from an electroanalytical measurement setup, and
an analyte ion measurement setup comprising:
   i. one or more, optionally solid-state, working electrodes, each of said, optionally solid-state, working electrodes comprising an ion-selective electrode, which is selective for an analyte ion, and
   ii. a reference electrode comprising an, optionally solid-state, ion-selective electrode, which is selective for the reference ion,
wherein the analyte ion measurement setup is an electroanalytical setup, such as a potentiometric setup,
and wherein the analyte ion measurement setup is being arranged for measuring one or more potential differences directly or indirectly between:
   i. each of the one or more working electrodes, and
   ii. the reference electrode.

In embodiments, each of the one or more working electrodes are solid-state electrodes and/or the reference electrode is a solid-state reference electrode.

The analyte ion measurement setup and the reference ion measurement setup may be rigidly connected and/or the apparatus may comprise an enclosure, such as a single enclosure, such as a single enclosure encompassing and surrounding the analyte ion measurement setup and the reference ion measurement setup.

The analyte ion measurement setup and the reference ion measurement setup may be arranged for probing the sample in the same measurement chamber in the apparatus. This may be advantageous for keeping a necessary amount of sample to a minimum since the sample need then only fill a single measurement chamber. Additionally, handling, rinsing, cleaning, etc., may be simplified because the sample need not be led to different measurement chambers.

According to an embodiment, there is presented an apparatus, wherein the reference ion measurement setup comprises an optical sensor, such as an optical for measuring the parameter indicative of a concentration of a reference ion, such as wherein the optical sensor is arranged for carrying out a measurement, which comprises measuring an optical parameter Po (such as absorption rate, reflectance, fluorescence, refractive index or color) which is dependent on pH and wherein a change dPₒ in optical parameter with a change dpH in pH, dPₒ/dpH has a local and/or global maximum within a pH interval of [7; 8], such as within a pH interval [7.2, 7.6], such as at or about 7.4. For example, the optical sensor may be an optical pH sensor, such as based on a reference ion sensitive indicator (changing one or more optical properties, such a absorption (e.g., in a wavelength region of ]400; 1200 [ nm, such as in a wavelength region of ]500; 1100[ nm, such as in a wavelength region of ]600; 1000[ nm, such as in a wavelength region of ]700; 900[ nm), depending on a concentration of the reference ion), such as based on a pH sensitive luminescent indicator.

A reference ion sensitive indicator may have a maximum or minimum of an optical property, such as an absorption maxima in a wavelength region with wavelengths being larger than 400 nm or larger than 500 nm or larger than 600 nm or larger than 700 nm and/or smaller than 1200 nm or smaller than 1100 nm or smaller than 1000 nm or smaller than 900 nm, such as within a wavelength region of ]400; 1200[ nm, such as in a wavelength region of ]500; 1100[ nm, such as in a wavelength region of ]600; 1000[ nm, such as in a wavelength region of ]700; 900[ nm. An advantage of a reference ion sensitive indicator having a maximum or minimum of an optical property within such wavelength region may be that haemoglobin absorption is minimal within 600-1000 nm and/or that interference on other sensors is limited (due to wavelengths in this region being sufficiently long not to interact/degrade entities (e.g., ionophores) in other sensors being present in the apparatus) and/or that scattering by lipid particles is limited (due to wavelengths in this region being sufficiently long to be less vulnerable to scattering by the relatively smaller lipid particles in a sample, such as in a biological sample, such as in a whole blood sample).

According to an embodiment, there is presented an apparatus, wherein the reference ion is any of:
a hydrogen ion, such as H⁺,
a sodium ion, such as Na⁺,
a potassium ion, such as K⁺,
a calcium ion, such as Ca²⁺,
a chloride ion, such as Cl⁻,
a magnesium ion, such as Mg²⁺, or
a hydrogen carbonate ion, such as HCO₃⁻.

Each of the listed reference ions is an ion naturally occurring within (human) whole blood. In this case, the reference ion may be measured directly on whole blood without the need for addition of ions to serve as reference ions.

According to an embodiment, there is presented an apparatus, further comprising:
A data processing device comprising a processor configured to:
   i. determine one or more concentrations of the one or more analyte ions in the sample based on:
      1. The concentration of the reference ion, and
      2. The one or more potential differences.

By implementing a data processing device, the determination of the one or one or more concentrations of the one or more analyte ions may be carried out in a faster, automated and/or more reliable manner.

According to an embodiment, there is presented an apparatus further comprising
a sample handling system comprising:
   a sample inlet, such as said sample inlet comprising an aspiration system,
   a measurement chamber (such as a volume of the sample handling system where the sample is placed while measurements of the parameter indicative of a concentration of a reference ion and/or the one or more potential differences are taking place), such as wherein both of the reference ion measurement setup (104) and the analyte ion measurement setup (105) being arranged for measuring on the sample while in the measurement chamber,
   one or more fluidic channels, such as microfluidic channels, fluidically connecting the sample inlet and the measurement chamber.

A possible advantage may be that sample handling is carried out in a more hygienic, safe, automated, reliable and/or repeatable way. By ‘sample handling system’ may generally be understood a system capable of receiving and handling the sample, such as bringing it from a sample inlet to a measurement chamber via one or more fluidic channels.

According to an embodiment, there is presented an apparatus, wherein the sample handling system is further comprising:
One or move valves, such as valves enabling segmented transportation of the sample, such as in the form of a sample plug, through the sample handling system, said valves optionally being controlled by a data processing device, such as the data processing device.

A possible advantage is that the valves may enable segmented transportation, such as transportation wherein a sample travels through at least part of the sample handling system (such as from sample inlet to measurement chamber) as a plug and/or without mixing with liquid in front of and/or behind the sample.

According to an embodiment, there is presented an apparatus, wherein an analyte ion concentration accuracy is lower than 20%, such as lower than 15%, such as lower than 10%, such as lower than 7%, such as lower than 5.4%, such as lower than 5%, such as lower than 3.5%, such as lower than 2.7%, for the analyte ion being a monovalent ion. An advantage of such low (where ‘low’ accuracy is understood as less deviation from a true value) accuracy is that a more accurate estimate of the concentration may be achieved, which may in turn enable improved diagnosis, improved assessment of treatment effect or improved estimates of physiological and/or nutritional conditions of a subject (such as patient or person from whom a blood sample was drawn). ‘Accuracy’ is understood as is common in the art, such as an error that will exist between the actual (‘true’) value, such as in this context a true concentration of the analyte ion, and a measured value, such as in this context a concentration of the analyte ion at the output of the apparatus and/or as determined by the apparatus.

According to an embodiment, there is presented an apparatus, wherein the apparatus, such as the entire apparatus, enables determining one or more concentrations of one or more analyte ions in a sample based on the more or more potential differences, with an accuracy with respect to one or more true concentrations, being lower than 20%, such as lower than 15%, such as lower than 10%, such as lower than 7%, such as lower than 5.4%, such as lower than 5%, such as lower than 3.5%, such as lower than 2.7%, for the one or more analyte ions each being a monovalent ion. It is to be understood, that the apparatus, such as the entire apparatus, i.e., e.g., determination of the analyte ion concentration based on both of the parameter indicative of a concentration of a reference ion and the potential difference being indicative of the concentration of the analyte ion, enables the low accuracy in terms of determining analyte ion concentration. Furthermore, it is to be understood that accuracy is determined relative to a true value of the concentration.

According to an embodiment, there is presented an apparatus, wherein an accuracy of a reference potential as determined by the (optionally solid state) reference electrode (i.e. total accuracy—i.e., taking into account accumulated error—of both the reference ion measurement setup and the reference electrode, which is selective for the reference ion) is lower than 10%, such as lower than 7.5%, such as lower than 5%, such as lower than 3.5%, such as lower than 2.7%, such as lower than 2.5%, such as lower than 1.75%, such as lower than 1.35%, for the analyte ion being a monovalent ion. An advantage of such low (where ‘low’ accuracy is understood as less deviation from a true value) accuracy of the reference potential, i.e., of the reference ion measurement setup and the reference electrode, is that a more accurate estimate of the reference potential is provided, which may in turn enable improved accuracy with respect to the concentration of the analyte ion, which may in turn enable improved diagnosis, improved assessment of treatment effect or improved estimates of physiological and/or nutritional conditions of a subject (such as patient or person from whom a blood sample was drawn). ‘Accuracy’ is understood as is common in the art, such as in this context as an error that will exist between the actual (‘true’) value of the reference potential (such as a “true” potential difference between a potential in a bulk part of the sample and within the conducting, internal electrode within the reference electrode) and a reference potential as determined based on the concentration of the reference ion as derived from the parameter indicative of the concentration of the reference ion as determined by the reference ion measurement setup and the potential at the reference electrode (such as the potential subtracted from the potential difference between the working electrode and the reference electrode to obtain a potential difference between a potential in a bulk part of the sample and within the conducting, internal electrode within the working electrode).

According to an embodiment, there is presented an apparatus, wherein
An analyte ion concentration accuracy is lower than 20%, such as lower than 15%, such as lower than 10%, such as lower than 7%, such as lower than 5.4%, such as lower than 5%, such as lower than 3.5%, such as lower than 2.7%, for the analyte ion being a monovalent ion, and
The fraction of the analyte ion concentration accuracy originating from the reference ion measurement setup and the reference electrode is less than 50%, such as less than 40%, such as less than 30%, such as less than 20%, such as less than 10%, such as wherein an analyte ion concentration accuracy is lower than 10% and the fraction of the analyte ion concentration accuracy originating from the reference ion measurement setup and the reference electrode is less than 30%, such as less than 25%, such as less than 20%. An advantage of this may be that an analyte ion concentration accuracy lower than 10% is sufficiently accurate for most purposes and the relatively low fraction originating from the reference ion measurement setup and the reference electrode leaves room for realistic accuracies (or lack thereof) originating from other sources, such as from the ion selective working electrode.

An advantage of such low (where ‘low’ accuracy is understood as less deviation from a true value) accuracy is that a more accurate estimate of the concentration may be achieved, which may in turn enable improved diagnosis, improved assessment of treatment effect or improved estimates of physiological and/or nutritional conditions of a subject (such as patient or person from whom a blood sample was drawn). ‘Accuracy’ is understood as is common in the art, such as an error that will exist between the actual (‘true’) value, such as concentration of the analyte ion, and the indicated value, such as concentration of the analyte ion, at the output of the apparatus and/or as determined by the apparatus.

According to an embodiment, there is presented an apparatus, wherein a distance (such as a centre-to-centre distance) between:
each of the one or more working electrodes, and
the reference electrode,
   is equal to or less than 10 mm, such as equal to or less than 5 mm, such as equal to or less than 3 mm, such as equal to or less than 1 mm, such as equal to or less than 1 mm. A possible advantage is that (only) a relatively small sample volume is required. Another possible advantage is that less (idle) time has to pass between measurements on different samples, e.g., in case of employing a sample chamber, which can be rinsed faster (e.g., due to the sample chamber volume being smaller).

According to an embodiment, there is presented an apparatus wherein a slope of a reference ion half-cell is at least 10%, such as at least 25%, such as at least 50%, such as at least 75%, such as at least 80%, such as at least 90%, such as at least 95%, such as at least 97%, with respect to a theoretical slope of the reference ion according to the Nernst equation. An advantage of having a relatively large slope may be that a normal, well-functioning, off-the-shelves ISE (which could be employed as working electrodes themselves) could be utilized as reference electrodes (because the reference ion measurement setup takes enables taking the slope/sensitivity into account). By slope of a reference ion half-cell is understood a constant of proportionality between measured potential and (logarithm to) the reference ion concentration, such as wherein a measured electrode potential, E, is related to the activity of an ionic species by the Nernst equation:

E=E₀+2.3xRT/(nF)log A

Where E₀=a constant for a given cell, R=the gas constant, T=the temperature in Kelvin, n=the ionic charge, F=the Faraday constant, A is activity and x is a factor taking into account that the slope factor may deviate from a factor given by the expression RT/nF, which may be termed the Ideal Slope Factor. For example, when measuring potassium ions, (i.e., n=+1), the slope factor at 298K (25° C.) has a value of 59.16 mV and the slope factor at 37° C. has a value of 61.54 mV. For example for x=0.1, a slope of the reference ion half-cell is 10% with respect to a theoretical (Ideal) slope of the reference ion according to the Nernst equation.

According to a third aspect, there is presented use of an apparatus according to the second aspect, wherein said apparatus is being used for measuring one or more potential differences being indicative of one or more concentrations of one or more analyte ions in a sample, such as the sample being a liquid whole blood sample, such as used for determining one or more concentrations of the one or more analyte ions in the sample, such as based on the concentration of the reference ion and the one or more potential differences.

In the context of point-of-care measurement systems (in the art also referred to as ‘bedsite’ systems) and laboratory environments alike, blood gas analysis is oftentimes undertaken by users, such as nurses, who may not be users trained in use of, e.g., blood gas analyzers.

According to a fourth aspect of the invention (or an embodiment of the third aspect), there is presented use of a use of an apparatus according to the second aspect of the invention for point-of-care (POC) analysis, such as determination of one or more concentrations of one or more analytes, of a sample, such as a sample being a liquid whole blood sample.

POC measurement is also referred to as ‘bed site’ measurement in the art. In the present context, the term ‘point-of-care measurement’ should be understood to mean measurements which are carried out in close proximity to a patient, i.e., measurements that are not carried out in a laboratory. Thus, according to this embodiment, the user of the apparatus, such as the apparatus being a blood gas analyzer, performs measurement of a whole blood sample in a handheld blood sample container in the proximity of the patient, from whom the blood sample is taken, e.g. in the hospital room or ward accommodating the patient's bed, or in a nearby room of the same hospital department. In such use, the level of expertise of the user oftentimes varies from novice to experienced, and the capability of the blood gas analyzer to automatically output instructions matching each individual user's skills on the basis of sensor input is thus particularly beneficial in such environments.

The first, second, third and fourth aspect of the present invention may each be combined with any of the other aspects. These and other aspects of the invention will be apparent from and elucidated with reference to the embodiments described hereinafter.

### BRIEF DESCRIPTION OF THE FIGURES

The method, apparatus and use according to the invention will now be described in more detail with regard to the accompanying figures. The figures show one way of implementing the present invention and is not to be construed as being limiting to other possible embodiments falling within the scope of the attached claim set.

Preferred embodiments of the invention will be described in more detail in connection with the appended drawings, and more particularly:

FIG. 1 is a schematic illustration of an apparatus 100,

FIG. 2 shows a method 200,

FIG. 3 shows an example of an electrochemical measurement setup 305 and a reference ion measurement setup 304,

FIG. 4 shows an enlarged view of the conducting, internal electrode 366 and the membrane 368,

FIG. 5 shows an overview of (deviation/error) sums in a graph with systems 1-10 (each formed by an NPT7 instrument 104, 304 and an ABL725 instrument) and the four different measurements (“#m. 1”, “#m. 2”, etc.) for each system.

### DETAILED DISCLOSURE OF THE INVENTION

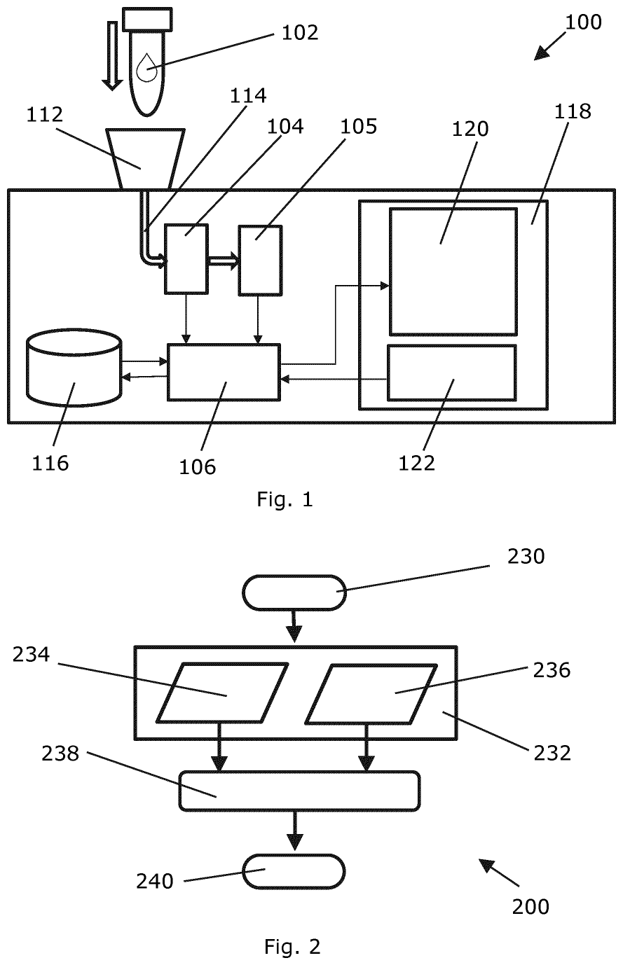

FIG. 1 is a schematic illustration of an apparatus 100 for measuring one or more potential differences being indicative of one or more concentrations of one or more analyte ions in a sample 102, such as the sample being a liquid whole blood sample, comprising:
a reference ion measurement setup 104, 304, being arranged for measuring 234 a parameter indicative of a concentration of a reference ion, wherein the reference ion measurement setup is different from an electroanalytical measurement setup, such as being an optical sensor, and
an analyte ion measurement setup 105, 305 comprising:
   i. one or more, optionally solid-state, working electrodes 352, each of said, optionally solid-state, working electrodes 352 comprising an ion-selective electrode, which is selective for an analyte ion,
   ii. a reference electrode 350 comprising an, optionally solid-state, ion-selective electrode, which is selective for the reference ion,
wherein the analyte ion measurement setup is an electroanalytical setup, such as a potentiometric setup,
and wherein the electrochemical measurement setup 105, 305 is being arranged for measuring 236 one or more potential differences directly or indirectly between:
   iii. each of the one or more working electrodes 352, and
   iv. the reference electrode 350.

The reference ion measurement setup 104, 304 and the electrochemical measurement setup 105, 305 are depicted as being comprised at or within separate measurement chambers, yet in alternative embodiments they could both probe the sample in the same measurement chamber 354.

The reference ion measurement setup 104, 304 in the schematic example comprises an optical, such as an optical pH sensor, and is further comprising
A data processing device 106 comprising a processor configured to:
   i. determine 238 one or more concentrations of the one or more analyte ions in the sample based on:
      1. The concentration of the reference ion,
      2. The one or more potential differences.

The apparatus in the schematic illustration in FIG. 1 furthermore shows a sample handling system comprising:
a sample inlet 112,
a measurement chamber 354,
one or more fluidic channels 114, such as microfluidic channels, fluidically connecting the sample inlet and the measurement chamber(s).

The depicted device furthermore comprises a digital storage device 116 (e.g., for storing data related for control of the data processing device and/or for calculations), a user interface 118, wherein the user interface comprises an output unit 120 arranged for visually outputting information relevant for operating the apparatus and/or information representative of one or more concentrations of the one or more analyte ions in the sample 102 (which output unit in the depicted embodiment is a display unit) and an input unit 122 (such as a keyboard, e.g., for providing information to the apparatus regarding the identity of a sample 102). The thin-line arrows indicate flow of information, such as a parameter indicative of a concentration of a reference ion flowing from the reference ion measurement setup 104, 304 to the data processing device 106 and one or more potential differences flowing from electrochemical measurement setup 105, 305 to the data processing device 106, one or more concentrations of the one or more analyte ions in the sample flowing from the data processing device 106 to the user interface 118 (and more particularly the output unit 120), user input flowing from the input unit 122 to the data processing device 106.

FIG. 2 shows a method 200 (starting at block 230, ending at block 240) of measuring one or more potential differences being indicative of one or more concentrations of one or more analyte ions in a sample 102 optionally being a liquid whole blood sample, and optionally furthermore determining the one or more concentrations of the one or more analyte ions, said method comprising,
Optionally providing 232 the sample 102 to a sample inlet 112 of an apparatus 100 as described in FIG. 1 and/or according to the second aspect of the invention,
Measuring 234 with a reference ion measurement setup 104, 304 a concentration of a parameter indicative of a reference ion in the sample 102, wherein the reference ion measurement setup 104 is different from an electroanalytical measurement setup, and optionally furthermore determining the reference ion concentration,
Measuring 236 with an analyte ion measurement setup one or more potential differences directly or indirectly between:
   iii. each of one or more working electrodes 352, such as one or more solid-state working electrodes, each of said working electrodes comprising an ion-selective electrode, which is selective for an analyte ion, and
   iv. a reference electrode 350, such as a solid-state reference electrode, which is selective for the reference ion,
wherein the analyte ion measurement setup is an electroanalytical setup, such as a potentiometric setup, and
Optionally determining 238 the one or more concentrations of the one or more analyte ions.

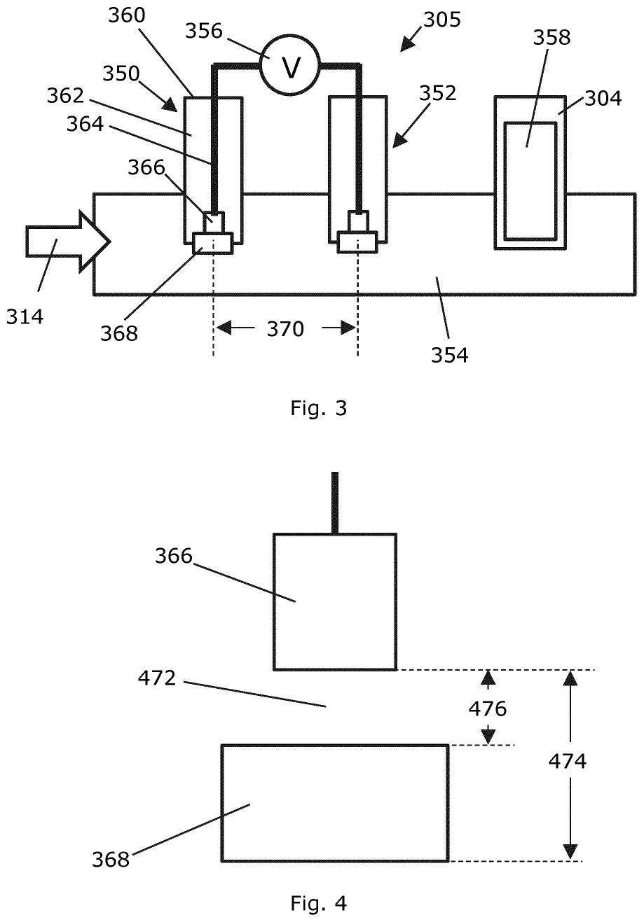

FIG. 3 shows an example of an electrochemical measurement setup 105, 305 and a reference ion measurement setup 104, 304. More particularly, the figure shows an electrochemical measurement setup 105, 305 comprising a reference electrode 350 and a working electrode 352 partially within a measurement chamber 354, a (high-impedance) voltmeter 356 electrically between the reference electrode 350 and the working electrode 352 and an (inlet) fluidic channel 314 arranged for allowing leading sample into the measurement chamber 354. The parts of the reference electrode 350 and the working electrode 352 are similar, but explained for simplicity only for the reference electrode 350: A casing 360, which comprises an insulating encapsulation material 362, which surrounds a conductor 364 electrically connected to a conducting, internal electrode 366, which is disposed on the other side of a membrane 368 with respect to the interior of the measurement chamber 354 (where the sample may be present during use). A centre-to-centre distance 370 between the reference electrode 350 and the working electrode 352 is also shown.

FIG. 3 also shows a reference ion measurement setup 104, 304 in the form of an optical pH sensor being partially within the measurement chamber 354 and comprising an optical analysis unit 358, which may comprise one or more light sources and one or more light detectors and optionally one or more optical filters.

FIG. 4 shows an enlarged view of the conducting, internal electrode 366 and the membrane 368 (such as of a working electrode 352 or a reference electrode 350). The figure furthermore shows that there might be a gap 472 between the conducting, internal electrode 366 and the membrane 368. Still further the figure indicates a distance 474 from the conducting, internal electrode 366 to the opposite side of the membrane 368, which is also a distance between the conducting, internal electrode 366 and the sample 102 (or the measurement chamber 354 where the sample 102 may be present during use). Still further, the figure indicates a distance 476 from the conducting, internal electrode 366 to the membrane 368.

### EXAMPLES

A human (non-smoker) blood sample 102 was adjusted to a total haemoglobin concentration of 15 g/dL and to a pH value of approximately 7.6 with gases ensuring oxygen saturation (SAT100), wherein the CO2 level in the gas controls the pH.

pH measurements of the blood sample 102 were carried out 5 ABL725 (Radiometer, Copenhagen, Denmark) electroanalytical blood gas analysers. Measurements were repeated four times on each ABL725 electroanalytical blood gas analyser. Results can be seen in TABLE I (with n in “ABL725-n” indicating number n of instrument).

TABLE I

| #mea- | | | | | |
|---|---|---|---|---|---|
| surement | ABL725-1 | ABL725-2 | ABL725-3 | ABL725-4 | ABL725-5 |
| 1 | 7.663 | 7.668 | 7.663 | 7.67 | 7.663 |
| 2 | 7.657 | 7.662 | 7.659 | 7.666 | 7.66 |
| 3 | 7.653 | 7.658 | 7.656 | 7.662 | 7.655 |
| 4 | 7.649 | 7.654 | 7.652 | 7.658 | 7.651 |

pH measurements 234 of the blood sample 102 were additionally carried out with 10 NPT7 (Radiometer, Copenhagen, Denmark) optical blood gas analysers 104, 304. Measurements were repeated four times on each NPT7 optical blood gas analyser. Results can be seen in TABLE II and TABLE III (with “#m.” indicating number of measurement and n in “NPT-n” indicating number of instrument) where the entries in Table II each indicate the discrete NPT7 measurement and entries in Table III each indicate the deviation or error from the average value as determined by the five ABL725 measurements on the same round of measurements in order to remove interference from a drift in the sample 102 (by degassing of CO₂ and a subsequent pH change).

TABLE II

| #m. | NPT7-1 | NPT7-2 | NPT7-3 | NPT7-4 | NPT7-5 | NPT7-6 | NPT7-7 | NPT7-8 | NPT7-9 | NPT7-10 |
|---|---|---|---|---|---|---|---|---|---|---|
| 1 | 7.681 | 7.674 | 7.685 | 7.687 | 7.678 | 7.684 | 7.672 | 7.683 | 7.691 | 7.680 |
| 2 | 7.682 | 7.677 | 7.674 | 7.686 | 7.669 | 7.682 | 7.679 | 7.681 | 7.677 | 7.670 |
| 3 | 7.673 | 7.654 | 7.671 | 7.682 | 7.662 | 7.672 | 7.657 | 7.674 | 7.678 | 7.662 |
| 4 | 7.668 | 7.668 | 7.667 | 7.676 | 7.671 | 7.659 | 7.660 | 7.673 | 7.661 | 7.662 |

TABLE III

| #m. | NPT7-1 | NPT7-2 | NPT7-3 | NPT7-4 | NPT7-5 | NPT7-6 | NPT7-7 | NPT7-8 | NPT7-9 | NPT7-10 |
|---|---|---|---|---|---|---|---|---|---|---|
| 1 | 0.0153 | 0.0083 | 0.0194 | 0.0218 | 0.0127 | 0.0189 | 0.0065 | 0.0175 | 0.0256 | 0.0147 |
| 2 | 0.0213 | 0.016 | 0.0127 | 0.0252 | 0.0081 | 0.021 | 0.0182 | 0.02 | 0.0162 | 0.0095 |
| 3 | 0.0166 | −0.0024 | 0.0144 | 0.025 | 0.0049 | 0.0154 | −0.0003 | 0.017 | 0.0209 | 0.0056 |
| 4 | 0.0148 | 0.0153 | 0.0142 | 0.0233 | 0.0185 | 0.0058 | 0.007 | 0.0201 | 0.0081 | 0.009 |

For assessing an impact on accuracy from replacing a fully electroanalytical method and apparatus with an apparatus 100 and method 200 according to embodiments of the invention 40 sums or errors were formed, with each sum being a sum of an error from an NPT7 measurement and an error from an ABL725 measurement (where the ABL725 dataset was duplicated to match the double-sized NPT7 dataset).

Assuming (for the purpose or providing a conservative estimate) that a fully electroanalytical method and setup would be relying on a perfect reference electrode 352, the impact on accuracy introduced with embodiments of the present invention could be estimated as the sum of the error on the optical pH measurement 234 of the NPT7 instrument 104, 304 (corresponding to the error introduced by the reference ion measurement setup) and the error introduced by the solid-state ion-selective pH electrode of the ABL725 instrument (corresponding to the error introduced by the reference electrode of the analyte ion measurement setup). It is noted, that depending on the signs of these errors, they may add up or (fully or partially) cancel each other.

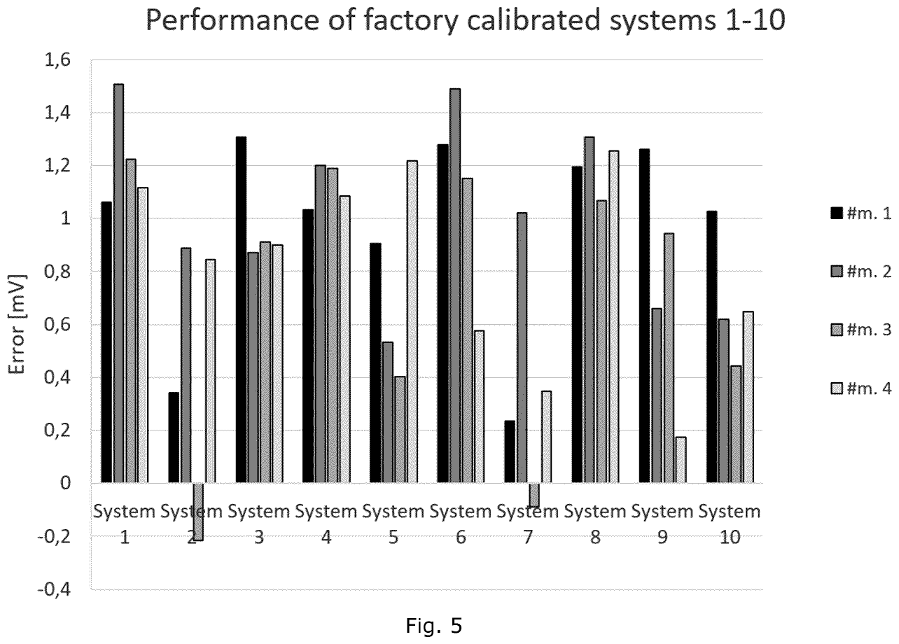

FIG. 5 shows an overview of these sums in a graph with systems 1-10 (each formed by an NPT7 instrument 104, 304 and an ABL725 instrument) and the four different measurements (“#m. 1”, “#m. 2”, etc.) for each system. It can be seen that all errors are below 1.60 mV with the errors generally being on the order of 1.0 mV with an average of the absolute values being 0.888 mV. For a Nernst factor of 60 mV this would correspond to an accuracy impact on an analyte ion measurement of 100%*(10^{(0.888 mV/60 mv)}−1)=3.47% for monovalent ions and 6.93% for divalent ions. Correcting for a mean value of the system, these values can be lowered to 2.7% and 5.4% mono- and divalent ions respectively. Assuming that an analyte ion selective working electrode introduces an error of the same order of magnitude or smaller, an accuracy of the method or apparatus according to the invention may be estimated to be somewhere between 2.7-5.4% for monovalent analyte ions and 5.4-10.8% for divalent analyte ions.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A method of measuring one or more potential differences being indicative of one or more concentrations of one or more analyte ions in a sample, said method comprising:
measuring with a reference ion measurement setup a parameter indicative of a concentration of a reference ion in the sample, wherein the reference ion measurement setup is different from an electroanalytical measurement setup, and
measuring with an analyte ion measurement setup the one or more potential differences being indicative of the one or more concentrations of the one or more analyte ions in the sample directly or indirectly between:
i. each of one or more working electrodes, each of said working electrodes comprising an ion-selective electrode, which is selective for an analyte ion, and
ii. a reference electrode, which is selective for the reference ion,
wherein the analyte ion measurement setup is an electroanalytical setup.

2. The method according to claim 1, wherein each of the one or more working electrodes are solid-state electrodes and/or wherein the reference electrode is a solid-state reference electrode.

3. The method according to claim 1, further comprising determining one or more concentrations of the one or more analyte ions in the sample based on:
i. the concentration of the reference ion, and
ii. the one or more potential differences.

4. The method according to claim 1, wherein the one or more potential differences depend on the reference ion concentration in the sample.

5. The method according to claim 3, wherein determining the one or more concentrations of the one or more analyte ions in the sample is based on an expression reflecting and/or incorporating the dependency of the one or more potential differences on the reference ion concentration in the sample.

6. The method according to claim 1, wherein the sample is a liquid whole blood sample.

7. The method according to claim 1, further comprising aspirating the sample, thereby creating an aspirated portion of the sample, and wherein each of measuring with the reference ion measurement setup and measuring with the analyte ion measurement setup is done on the aspirated portion of the sample.

8. The method according to claim 1, wherein measuring with the reference ion measurement setup a concentration of the reference ion in the sample, comprises an optical measurement.

9. The method according to claim 1, wherein the reference ion is the hydrogen ion, the sodium ion, or the potassium ion.

10. The method according to claim 8, wherein the optical measurement comprises measuring an optical parameter Pₒ which is dependent on pH and wherein a change dPₒ in optical parameter with a change dpH in pH, dPₒ/dpH has a local and/or global maximum within a pH interval of [7; 8].

11. An apparatus for measuring one or more potential differences being indicative of one or more concentrations of one or more analyte ions in a sample, comprising:
a reference ion measurement setup, being arranged for measuring a parameter indicative of a concentration of a reference ion, wherein the reference ion measurement setup is different from an electroanalytical measurement setup, and
an analyte ion measurement setup comprising:
i. one or more, optionally solid-state, working electrodes, each of said, optionally solid-state, working electrodes comprising an ion-selective electrode, which is selective for an analyte ion, and
ii. a reference electrode comprising an, optionally solid-state, ion-selective electrode, which is selective for the reference ion,
wherein the analyte ion measurement setup is an electroanalytical setup,
and wherein the analyte ion measurement setup is being arranged for measuring one or more potential differences directly or indirectly between:
v. each of the one or more working electrodes, and
vi. the reference electrode.

12. The apparatus according to claim 11, wherein the reference ion measurement setup comprises an optical sensor.

13. The apparatus according to claim 11, further comprising:
a data processing device comprising a processor configured to determine one or more concentrations of the one or more analyte ions in the sample based on: the concentration of the reference ion, and the one or more potential differences.

14. The apparatus according to claim 11, further comprising a sample handling system comprising:
a sample inlet,
a measurement chamber, and
one or more fluidic channels, fluidically connecting the sample inlet and the measurement chamber.

15. The apparatus according to claim 11, wherein an accuracy is lower than 20%, for the analyte ion being a monovalent ion.

16. The apparatus according to claim 11, wherein the apparatus enables determining one or more concentrations of one or more analyte ions in a sample based on the more or more potential differences, with an accuracy with respect to one or more true concentrations, being lower than 20% for the one or more analyte ions each being a monovalent ion.

17. The apparatus according to claim 11, wherein a distance between: each of the one or more working electrodes, and the reference electrode is equal to or less than 10 mm.

18. (canceled)

19. The method according to claim 10, wherein the dPₒ/dpH has a local and/or global maximum within a pH interval of [7.2, 7.6].

20. The method according to claim 19, wherein the dPₒ/dpH has a local and/or global maximum at a pH of about 7.4

21. The apparatus according to claim 15, wherein the accuracy is lower than 7% for the analyte ion being a monovalent ion.
